(19) United States
(12) Patent Application Publication
(10) Pub. No.: US 2006/0034883 A1
(43) Pub. Date: Feb. 16, 2006

(54) Composite stent with polymeric covering and bioactive coating

(76) Inventors: Mai Huong Dang, Palo Alto, CA (US); Phillip Chiu, San Francisco, CA (US); Leon V. Rudakov, Belmont, CA (US); Kevin T. Larkin, Menlo Park, CA (US); Mir A. Imran, Los Altos Hills, CA (US)

Correspondence Address: PERKINS COIE LLP, P.O. BOX 2168, MENLO PARK, CA, 94026, US

(21) Appl. No.: 10/499,773

(22) PCT Filed: Dec. 21, 2001
(86) PCT No.: PCT/US01/49635

§ 371 Date: May 20, 2005

(51) Int. Cl. A61M 31/00

(52) U.S. Cl. 424/422; 604/500

(57) ABSTRACT

A composite expandable stent for delivery into a vessel carrying blood comprising an expandable support frame having first and second end portions. A porous imprevious polymer sleeve having inner and outer surfaces extending over the support frame. A coating is disposed on at least one of the inner and outer surfaces of the polymer sleeve for enhancing endothelial cell growth on the device and polymer sleeve. The stent can be cylindrical or tapered.

### FIELD OF THE INVENTION

This invention relates to a composite expandable device with a polymeric covering on the device and a bioactive coating on device and the polymeric covering, a delivery apparatus and a method.

### BACKGROUND OF THE INVENTION

Saphenous vein grafts have heretofore been utilized for bypassing occluded arterial blood vessels in the heart. Because they are vein tissue rather than arterial tissue, they have different characteristics and generally do not function well long term as arterial vessels. Saphenous bypass veins are less muscular and are generally quite flimsy and compliant. When these saphenous vein grafts become diseased with age, stenoses and obstructive deposits which are cheesy or buttery in consistency and which are very malleable are formed which cannot be treated effectively with interventional catheter procedures even when followed with a stent implant. The plaque material forming the stenosis tends to ooze through the stent and reoccludes flow passage through the stent and the saphenous vein graft. Other vascular obstructions, such as in femoral and popliteal vessels and in carotids as well as in native coronary arteries also suffer from occlusions. In many of these cases, plaque proliferates through the stents when stents are deployed in the vessels. Therefore a great need exists for a new and improved device and method to provide a lasting therapeutic relief in such situations.

### SUMMARY OF THE INVENTION

In general, it is an object of the present invention to provide a composite expandable device with a substantially impervious polymeric covering thereon with a bioactive coating on the device and covering and a method for using the same which can be utilized for treating occlusions or partial occlusions in blood vessels and particularly saphenous vein grafts. In one embodiment, the polymeric covering is impervious. In another embodiment, the polymeric covering is porous.

Another object of the invention is to provide a device of the above character which will provide a lasting therapeutic solution to the occurrence of plaque in stents in saphenous vein grafts.

Another object of the invention is to provide a device of the above character which can be used for repaving with endothelial cells the portion of the vessel being treated. In one embodiment, the coating is a cell adhesion peptide. In a related embodiment, the coating has the amino acid sequence presented as SEQ ID NO: 1.

Another object of the invention is to provide a device of the above character which has the physical characteristics which substantially match or mimic the physical characteristics of blood vessels.

Another object of the invention is to provide a device of the above character in which a uniformly distributed structural support is provided for the polymeric covering.

Another object of the invention is to provide a device of the above character which is very flexible and can bend axially to accommodate the tortuosity of blood vessels.

Another object of the invention is to provide a device of the above character which can be placed in tandem with another similar device in a vessel to treat a long stenosis in a vessel.

Another object of the invention is to provide a device for delivery into a vessel carrying a blood comprising a polymer sleeve having inner and outer surfaces, and a coating disposed on and attached to at least one of the inner and outer surfaces of the polymer sleeve for enhancing endothelial cell growth on the polymer sleeve.

Additional objects and features of the invention will appear from the following description in which the preferred embodiments are set forth in detail in conjunction with the accompanying drawings.

### BRIEF DESCRIPTION OF THE FIGURES

FIG. 1 is a side elevational view of a composite expandable device with a polymeric covering and a bioactive coating thereon, with certain portions broken away, mounted on a balloon delivery catheter.

FIG. 2 is a cross-sectional view taken along the line 2-2 of FIG. 1.

FIG. 3 is a cross-sectional view taken along the line 3-3 of FIG. 1.

FIG. 4 is an enlarged detailed view of the balloon with the composite expandable device mounted thereon shown in FIG. 1.

FIG. 5 is a plan view of the expandable device which has been split apart longitudinally and spread out to show its construction.

FIG. 6 is a side elevational view of another embodiment of a composite expandable device with polymeric covering and bioactive coating thereon which is tapered and is carried by a tapered balloon for expansion and delivery.

FIG. 7 is a schematic illustration of a heart showing the manner in which a saphenous vein graft is treated utilizing the composite expandable device of the present invention.

FIG. 8 is an enlarged detailed view showing the docking of a tapered composite expandable device being docked with a cylindrical composite expandable device.

FIG. 9 is a flow chart of one embodiment of the present invention.

FIG. 10 is a cross-sectional view of a medical device having a surface treated in accordance with one embodiment of the present invention.

### DETAILED DESCRIPTION OF THE INVENTION

In general, the composite expandable device incorporating the present invention is for delivery into a vessel carrying blood and comprises an expandable support frame having first and second ends. An impervious or porous polymer sleeve extends over the support frame and may leave the first and second ends of the support frame exposed. A bioactive coating is provided on one or both of the inner and outer surfaces of the polymer sleeve and the frame for enhancing endothelial cell growth on the blood contact surfaces of the polymer sleeve and frame.

More in particular, the composite expandable device 11 as shown is mounted on a delivery apparatus 12 which consists of an expandable balloon 13 mounted on the distal extremity of a shaft or catheter 14 and having a wye fitting 16 mounted on the proximal extremity. The shaft or catheter 14 is provided with a central lumen 17 which is adapted to receive a conventional guide wire 18 through a port 19 provided in the fitting 16. The catheter shaft 14 is provided with a concentric lumen 21 which is in communication with a port 22 of the fitting 16. The lumen 21 extends through the balloon 13 and an opening (not shown) is provided in the shaft 14 within the balloon for inflating and deflating the balloon.

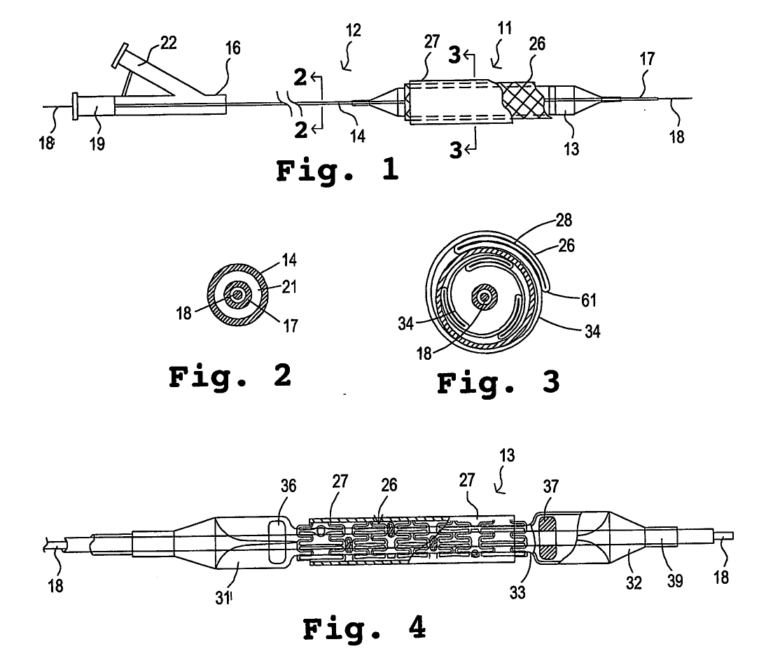

The composite expandable device 11 consists of an expandable frame 26 which has a polymeric sleeve 27 covering the same. The sleeve has folds 28 therein when the frame is in an unexpanded condition as shown in FIG. 4.

The expandable balloon 13 has a substantially continuous diameter and is provided with distal and proximal portions 31 and 32 and an intermediate portion 33 which serves as a working portion of the balloon, having a length which will accept the length of the composite device 11. The balloon 13 is provided with folds 34 when deflated as shown in FIGS. 1, 3 and 4. Radiopaque marker bands 36 and 37 are provided on the portion of the shaft 14 extending through the balloon 13 and are mounted in the distal and proximal portions 31 and 32 as shown adjacent to the intermediate portion 33. These marker bands 36 and 37 are within the distal and proximal portions 31 and 32 of the balloon 13 but have a diameter of the intermediate portion 33 with the composite expandable device 11 mounted on the intermediate portion 33 to serve as stops or abutments to prevent the composite expandable device 11 from inadvertently slipping off of the balloon 13 during positioning and deployment of the composite expandable device 11.

The frame 26 which forms a part of the composite expandable device 11 consists of a plurality of circumferentially spaced-apart elongated struts 41 having first and second ends 42 and 43. Foldable links 46 are secured to the first and second ends 42 and 43 and extend circumferentially of the frame 26 and serve in conjunction with the elongate struts to form a circular belt 47. As shown in FIG. 4, a plurality of serially-connected belts 47 are provided which are axially aligned with each other.

Sinusoidal-shaped end portions 48 and 49 are provided on opposite ends of the plurality of serially-connected belts 47. Interconnecting means 50 is provided for interconnecting the plurality of belts 47 and the end portions 48 and 49 so that the belts 47 and end portions 48 and 49 extend along an axis while permitting axial bending between the belts 47 and the end portions 48 and 49 while maintaining a constant length of the device 11. The means 50 consists of at least one strut 51 which is relatively short in length in comparison to the length of the elongate struts 41 and a plurality of S-shaped links 52. Thus, as shown in FIGS. 3 and 4, between each end portion and a belt and between adjacent belts there is provided a single strut 51 and two S-shaped links 52 all of which are spaced 120° apart the interconnecting means between adjacent belts and/or end portions are offset by 60°. Thus, with the construction shown in FIG. 4 there are provided four belts 47 and two end portions 48 and 49 with five sets of interconnecting means 50.

It can be seen that the length of the frame 26 can be readily increased or decreased by changing the number of belts 47 provided in the frame 26.

The frame 26 can be formed of a suitable material such as a metal or plastic. Suitable metals are stainless steel, titanium, and alloys thereof and other biocompatible metals. The plastic can be a polymer. Since the frame to be utilized in the composite expandable device is typically used in a saphenous vein graft, it need not have the radial strength normally required for stents placed in native arterial vessels. The frame 26 has been specifically designed to support the polymer sleeve 27 for use in saphenous vein graft to closely approximate mechanical properties of the saphenous vein graft. The same principles can be used for a composite device for arterial vessels and other blood vessels. Thus the frame 26 provides the necessary strength and consistency throughout its length while giving good flexibility throughout its length to accommodate movement of the saphenous vein graft.

As shown in FIG. 3, the polymer sleeve extends over substantially the entire length of the frame 26 but leaving end portions 48 and 49 substantially exposed for a purpose hereinafter described. The sleeve 27 typically is formed of a suitable polymer. One polymer found to be particularly satisfactory is PTFE which is supplied as a tube having a wall thickness ranging from 0.002″ to 0.010″ and preferably 0.003″ to 0.008″ and having a suitable original diameter as for example 2 to 4.5 mm. The expanded PTFE material should have a pore size, or internodal distance, of approximately 10 to 90 μm. Preferably the pore size or internodal distance is between about 40 to 70 μm. In addition in certain applications of this device, it may be desirable that the material be expandable from two to six times its original size yet retain elasticity properties to remain tightly over and in close engagement with the frame 26 prior to and after expansion. After placing the sleeve 27 over the working or intermediate portion 33 of the balloon, the sleeve 27 may be secured to the frame 26 during deployment as hereinafter described. To accomplish this, the sleeve 27 can be wrapped into a fold or a wing 28 and held in place along a line 61 (see FIG. 4) or tacked by spaced-apart heat seals (not shown) that are easily rupturable upon expansion of the frame 26. It has been found that such tacking by the use of heat seals on a fold or wing of the polymer sleeve 27 makes it easy for the balloon 13 when expanding to open the sleeve 27 without any significant additional balloon pressure being required.

With such a construction as shown in FIG. 3, the frame 26 which has been crimped onto the intermediate portion 33 of the balloon 13 and the sleeve 27 wrapped over onto the same and seamed into place will have an overall profile which has a diameter or size which is not greater than or desirably less than the diameters of the proximal and distal portions 31 and 32. Since the marker bands 36 and 37 have larger diameters than the intermediate portion 33 of the balloon 13, they will ensure that the composite expandable device consisting of the frame 26 and the sleeve 27 cannot inadvertently slip off of the balloon 13 during the procedure.

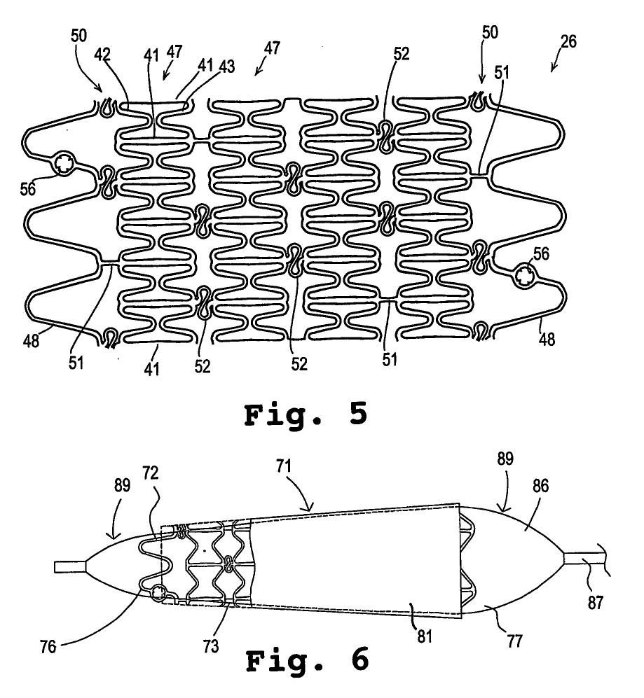

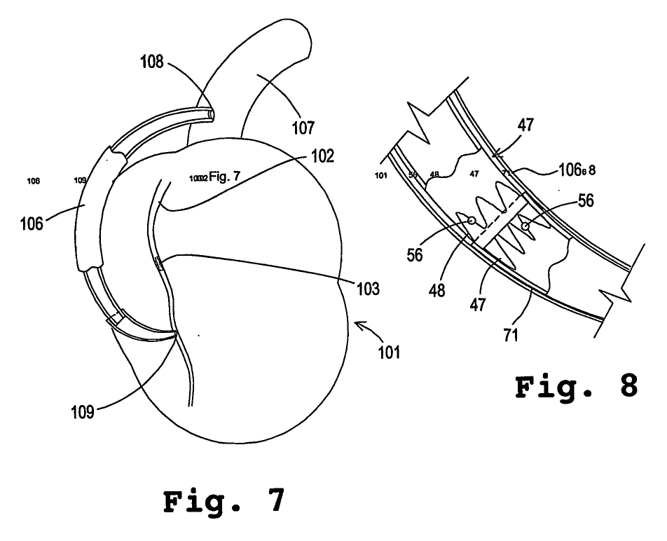

Another embodiment of a composite expandable device incorporating the invention is in the device 71 shown in FIG. 5. It is tapered rather than cylindrical to more closely approximate natural vessel geometry. In this device 71, a frame 72 is provided which is constructed in substantially the same manner as frame 26 but with the belts 73 increasing successively in circumference in one direction along the axis of the device 71 by providing foldable links 46 of successively greater lengths to provide the tapered construction shown in which one expandable end portion 76 has a lesser diameter than the other end portion 77. The means connecting the belts 73 and the end portions 76 and 77 are like the interconnecting means 50 hereinbefore described.

A tapered polymer sleeve 81 is provided on the exterior of the frame 72 while leaving the end portions 76 and 77 substantially exposed. A tapered balloon 86 is disposed within the frame 72 and is utilized for expanding the composite expandable device 71. The tapered balloon 86 is mounted on the distal extremity of a balloon shaft or catheter 87 and is constructed in the same manner as balloon shaft 14 and provides a delivery apparatus 89.

In order to provide a cell-friendly surface or surfaces on the sleeves 27 and 81, at least one surface of the outer and inner surfaces and preferably both inner and outer surfaces are treated in the following manner.

In general the method for treating a medical device having at least one surface exposed to tissue and/or blood and comprises the steps of subjecting the one surface to a low temperature plasma of an appropriate chemical agent to provide a plasma deposited layer having functional groups like amine, carboxylic, or hydroxyl groups covalently bound to the surface of the device. The plasma deposited layer is then subjected to a chemical treatment with multifunctional linkers/spacers which then become covalently bound with the plasma deposit layer. A bioactive coating is then covalently bound to spacers/linkers.

More in particular, the method as hereinafter described utilizes a plasma chamber (not shown) of the type as described in U.S. Pat. No. 5,643,580 well known to those skilled in the art and thus will not be described in detail. Typically the plasma utilized in the method of the present invention utilizes a low temperature or cold plasma produced by glow discharge. A low temperature plasma is created in an evacuated chamber refilled with a low pressure gas having a pressure on the order of 0.05 to 5 Torr and with the gas being excited by electrical energy usually in the radio frequency range. A glow discharge is created typically in the range of 2-300 watts for low power and 50-1000 watts for high power depending on the chamber volume.

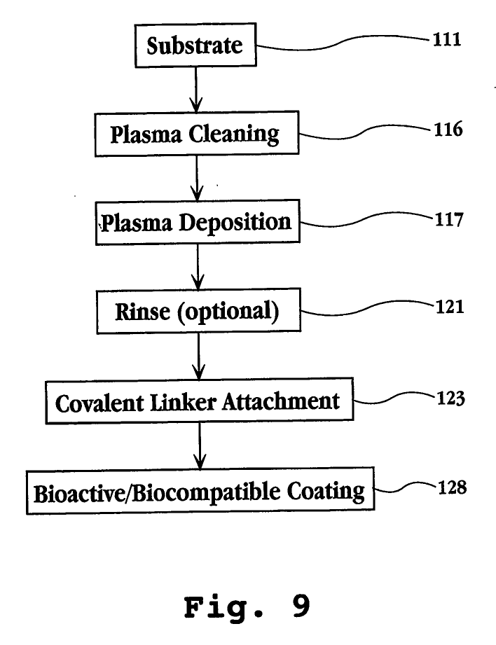

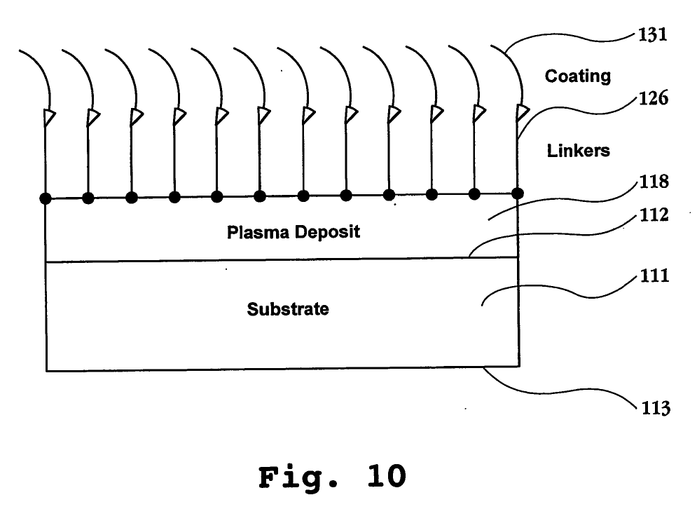

The steps for the method are shown in FIG. 9 for the treatment of a substrate 111 shown in FIG. 10 and having first and second surfaces 112 and 113. The substrate 111 is part of a medical implant or medical device that has at least one surface which is to be treated, such as one of the surfaces 112 and 113, to achieve desirable biological activities on that surface. The substrate 111 is formed of a suitable material such as a fluorinated thermoplastic or elastomer or more specifically, by way of example, PTFE. The latter material is particularly desirable where the medical implant or medical device is in the form of small-diameter vascular grafts. The substrate can also be formed of any polymer and polymer composites, metals and metal-polymer composites.

Let it be assumed that the surface 112 of the substrate 111 is to be treated in accordance with the method set forth in FIG. 9. The surface 112 is cleaned in an oxygen or air plasma as shown by step 116 in a relatively short period of time. The plasma cleaning process is an ablation process in which radiofrequency power, as for example 50-1000 watts, under a higher pressure e.g. 0.1 to 1.0 Torr at a high flow rate, as for example of at least 50 cc. per minute gas passing through the plasma chamber. Such a cleaning process can use oxygen, hydrogen alone, a mixture of oxygen with argon or nitrogen for a period of time of up to 5 minutes. Thus, a plasma of oxygen, air, or inert gases can be utilized for plasma cleaning.

Thereafter, the surface 112 after being cleaned as shown in step 116, is functionalized as shown in step 117 by subjecting the surface 112 to a pure gas or gas mixture plasma to assist in the deposition of functional groups on the surface 112 to provide a deposited layer 118 which is covalently bound to the surface 112. Other methods which can be utilized in place of the plasma deposition step 117 include a modification by irradiation with ultraviolet or laser light in the presence of organic amine or hydrazine. The plasma deposition step 117 used to achieve activation of the surface utilizes precursor gases which can include the following inorganic and organic compounds: NH₃ (ammonia), N₂H₄ (hydrazine) aliphatic amines, aliphatic alcohols, aliphatic carboxylic acids, allylamine, water vapor, allyl alcohol, vinyl alcohols, acrylic acid, methacrylic acid, vinyl acetate, saturated or unsaturated hydrocarbons and derivatives thereof. Precursors can be saturated (aliphatic amines, aliphatic alcohols, aliphatic acids) or unsaturated (allyl, vinyl and acrylated compounds). Employing unsaturated precursors or operating pulsed plasma (single mode or gradient) tend to preserve functional groups rather than form defragmentation products, having the potential of introducing a significantly higher percentage of reactive groups.

The deposition step 117 can be performed in continuous or pulsed plasma processes. The power to generate plasma can be supplied in pulsed form or can be supplied in graduated or gradient manner, with higher power being supplied initially, followed by the power being reduced or tapered towards the end of the plasma deposition process. For example, higher power or higher power on/off ratios can be utilized at the beginning of the step 117 to create more bonding sites on the surface 112 which results in stronger adherence between the substrate surface 112 and the deposited layer 118. Power is then tapered off or reduced as for example by reducing the power-on period to obtain a high percentage of functional groups on the surface 112.

The plasma deposition layer 118 created on the surface 112 has a thickness ranging from 5-1000 Å. By way of example this can be a layer derived from allylamine plasma. This plasma-assisted deposition typically is carried out at a lower power that ranges from 2-400 watts and typically from 5-300 watts depending upon the plasma chamber size, pressure and gas flow rate. This step 117 can be carried out for a period of time ranging from 30 seconds to 30 minutes while being sure that the reactive group created is preserved.

When it is desired to retain only those functional groups in the layer 118 which have established stable bonds to the substrate surface 112, as for example to a PTFE surface, an optional step 121 can be performed by rinsing or washing off loosely bound deposits with solvents or buffers. Thus, deposits which are merely adsorbed on the surface 112 are rinsed and washed off and the covalently bound deposits remain on the surface. Such a step helps to ensure that parts of the coating forming the layer 118 cannot thereafter be washed off by shear forces or ionic exchanges with blood flow passing over the surface.

Plasma-assisted deposition has been chosen because it is a clean, solvent-free process which can activate the most inert substrates like PTFE. Plasma produces high energy species, i.e., ions or radicals, from precursor gas molecules. These high energy species activate the surface 112 enabling stable bondings between the surface 112 and activated precursor gas. Allylamine has been chosen as a precursor for the plasma-assisted deposition step because it has a very low boiling point of 53° C., making it easy to introduce as a gas into the plasma chamber. By using allylamine, the desire is to have radicals created by the plasma occurring preferentially at C═C double bonds so that the free amine groups created are preserved for other reactions as hereinafter described. Also, it is believed to give a high yield of the desired primary amine group on the surface 112.

In the rinsing step 121, a solvent rinse such as dimethylsulfoxide (DMSO) is used for removing all of the allyamine deposit which has not been covalently bound to the surface 112, i.e. to remove any allylamine which has only been adsorbed on the surface. Another material such as dimethylformamide (DMF), tetrahydrofuran (THF) or dioxane can be utilized as a solvent rinse. In addition, for removing polar deposits, a buffer rinse can be utilized. As soon as the rinsing step 121 has been completed and the substrate 111 dried, wetting or surface tension measurement showed very hydrophilic PTFE (layer 118) completely wet with water. The presence of free amine groups can be visualized by tagging fluorescent probes reactive with amine groups. ATR-FTIR (attenuated total reflectance-fourier transform infrared) or ESCA (electron spectroscopy for chemical analysis) may give information about the presence of amine or nitrogen in layer 118, respectively.

Subsequently, in step 123, homo or hetero multifunctional linkers/spacers react and form stable linkages with the functional groups in layer 118 obtained by the plasma-assisted deposition process. This treatment in step 123 serves to provide linkers/spacers as represented by symbols 126 in FIG. 10 to improve accessibility of coating agents, as for example peptides and proteins, to functional groups on substrates. Vice versa, it is believed that the linkers 126 enhance the exposure of peptides and proteins to the environment. Also the linkers give peptides or proteins in the final coating more space and freedom to assume their natural conformations. As a result, the covalently bound coating agents are more likely to maintain their natural conformations and therefore their bioactivity.

By way of example, primary amine groups obtained after allylamine plasma react with succinic anhydride leading to a substrate covered by linkers 126 ended with COOH groups. Thus, the coverage with linkers 126 is less thrombogenic and more cell-friendly compared to the coverage with NH₂ rich layer 118. The linker/spacer attachment step 123 can also be utilized to introduce desirable functional groups which can readily react with the final coating agents. For example, COOH groups at the end of linker 126 can form stable amide linkage with NH₂ groups in cell-adhesion peptides and proteins, anti-inflammatory peptides, anti-thrombogenic peptides and proteins, growth factors, etc. The COOH groups can also form an ester linkage with OH groups in the anti-coagulant agent heparin. Taking the nature of the substrate, functional groups obtained after the plasma, the availability of functional groups and the size and nature of the final coating agents into consideration, the chemistry and size of the linkers may be selected. Multifunctional linkers usually have 2-20 carbon atoms in the backbone. They can be anhydrides of dicarboxylic acids, dicarboxylic acids, diamines, diols, or amino acids. Linkers can be just one molecule, a string of several molecules, such as a string of amino acids, a string of alternate dicarboxylic acids-diamines, dicarboxylic acids-diols or anhydrides-diamines. This chemical treatment step 123 hereinbefore described can also be characterized as one that introduces other desirable functional or activating groups.

Organic solvents which are miscible with water can be used as solubility enhancers to facilitate coupling efficiency between the plasma-treated substrate and the linkers (step 123) and/or coating agents (step 128) in an aqueous medium. DMSO, DMF or dioxane can be used as such solubility enhancers. They facilitate the contact between functional groups present in molecules of different hydrophilicity or hydrophobicity. After the corresponding functional groups present in molecules of different hydrophilicity or hydrophobicity. After the corresponding functional groups come close enough to each other, chemical reactions between them can occur. So, solubility enhancers in an aqueous solution can augment the binding reactions. The solubility enhancers may also enhance the accessibility of the linker/coating agents to the functional groups on porous surfaces.

After completion of the wet chemistry linker/spacer attachment step 123, the wetting behavior/surface tension of the resulting surface can be analyzed. Appropriate techniques, such as ESCA, SIMS, ATR-FTIR can be used to characterize the hydrophilic surface created in step 123. Fluorescent imaging of functional groups can also be carried out.

The bioactive/biocompatible coating step 128 can be carried out to provide the final layer of coating 131 on the surface 112 of the substrate 111 (as shown in FIG. 10). In this step, the available functional groups provided by the linkers 126, are used to covalently bind molecules of a bioactive/biocompatible agent, such as a cell-adhesion peptide P15 as hereinafter described, possessing desirable properties to the substrate surface 112 to provide the final resulting coating on the surface 112 as for example a PTFE surface. Of interest are bioactive/biocompatible coatings which, among others, can reduce foreign body reactions, accelerate the functioning and integration, as well as increase the long-term patency of implants. Such coatings can include cell adhesion peptides, proteins or components of extra-cellular matrix to promote cell migration and proliferation, leading to a rapid and complete coverage of the blood-contacting surface by a natural endothelial cell lining. Coatings with growth factors such as VEGF may lead to similar results. Non-adhesive coatings with polyethylene glycol derivatives are used for biocompatible hydrophilic surfaces as separation membranes, immuno barriers or surfaces free of platelet adhesion. Also, anti-thrombogenic coatings with hirudin, hirudin analogs, reversible and irreversible thrombin inhibitor peptides, or anti-coagulant coatings with heparin are desirable to reduce or prevent thrombosis formation at the implanting site. These local anti-thrombogenic or anti-coagulant coatings are more preferable than a systemic anti-coagulant treatment. Anti-inflammatory coatings can be used because occlusions may originate at inflamed sites. Anti-proliferative coatings are another way to reduce vessel occlusions by preventing smooth muscle cell proliferation.

The covalent immobilization of bioactive/biocompatible agents onto substrate members according to the present invention is generally non-reversible, i.e., the bioactive/biocompatible agent is not readily released from the functional group or surface-modifying group. However, multi-functional groups capable of selectively releasing an immobilized bioactive/biocompatible agent, including therapeutic drugs, have utility in receptor/ligand interactions, molecular identification and characterization of antibody/antigen complexes, and selective purification of cell subpopulations, etc. In addition, a selectively cleavable multifunctional linker affords predictable and controlled release of bioactive/biocompatible agents from the substrate.

Thus, the invention includes in one aspect a cleavable multi-functional linker. In this embodiment, selective release of the bioactive/biocompatible agent is performed by cleaving the spacer compound under appropriate reaction conditions including, but not limited to, photon irradiation, enzymatic degradation, oxidation/reduction, or hydrolysis, for example. The selective cleavage and release of immobilized agents may be accomplished using techniques known to those skilled in the art. See for example, Horton and Swaisgood, 1987; Wong, 1991; and U.S. Pat. No. 4,745,160, which is incorporated herein by reference. Suitable compounds for use as cleavable multifunctional linkers include, but are not limited to, polyhydroxyacids, polyanhydrides, polyamino acids, tartarates, and cysteine-linkers such as Lomant's Reagent.

Bioactive/biocompatible agents may be immobilized onto the substrate using bioconjugation techniques known to those skilled in the art. See Mosbach, 1987; Hermanson, et al., 1992; and Brinkley, 1992; for example. Mild bioconjugation schemes are preferred for immobilization of bioactive/biocompatible agents in order to eliminate or minimize damage to the structure of the substrate, the functional groups, the surface-modifying groups, and/or the bioactive/biocompatible agents.

Bioactive/biocompatible agents of the present invention are typically those that are intended to enhance or alter the function or performance of a particular substrate or alter the reactions and functions of the surrounding tissues. In one embodiment, biomedical devices for use in physiological environments are substrates contemplated by the present invention. In a particularly preferred embodiment, the bioactive/biocompatible group is selected from the group consisting of cell attachment factors, growth factors, antithrombotic factors, binding receptors, ligands, enzymes, antibiotics, and nucleic acids. The use of one bioactive/biocompatible agent on a substrate is presently preferred. However, the use of two or more bioactive/biocompatible agents on a substrate is also contemplated in one embodiment of the invention.

In a related embodiment, the invention includes a first bioactive/biocompatible agent that may be released slowly, and a second bioactive/biocompatible agent that may be released faster, e.g. by physical desorption. This combination would have an advantage in different phases in the course of disease treatment, wound healing, or incorporation of an implantable device. An exemplary slow release agent is released by hydrolysis of an ester bond formed between an OH group on the bioactive agent and the COOH formed on the substrate surface.

Desirable cell attachment factors include attachment peptides, as well as active domains of large proteins or glycoproteins typically 100-1000 kilodaltons in size, which in their native state can be firmly bound to a substrate or to an adjacent cell, bind to a specific cell surface receptor, and mechanically attach a cell to the substrate or to an adjacent cell. Attachment factors bind to specific cell surface receptors, and mechanically attach cells to the substrate or to adjacent cells. Such an event typically occurs within, well defined, active domains of the attachment factors. Factors that attach cells to the substrate are also referred to as substrate adhesion molecules herein. Factors that attach cells to adjacent cells are referred to as cell-cell adhesion molecules herein. In addition to promoting cell attachment, each type of attachment factor can promote other cell responses, including cell migration and differentiation. Suitable attachment factors for the present invention include substrate adhesion molecules such as the proteins laminin, fibronectin, collagens, vitronectin, tenascin, fibrinogen, thrombospondin, osteopontin, von Willibrand Factor, and bone sialoprotein, or active domains thereof. Other suitable attachment factors include cell-cell adhesion molecules, also referred to as cadherins, such as N-cadherin and P-cadherin.

Attachment factors useful in this invention typically comprise amino acid sequences or functional analogues thereof that possess the biological activity of a specific domain of a native attachment factor, with the attachment peptide typically being about 3 to about 20 amino acids in length. Native cell attachment factors typically have one or more domains that bind to cell surface receptors and produce the cell attachment, migration, and differentiation activities of the parent molecules. These domains consist of specific amino acid sequences, several of which have been synthesized and reported to promote the attachment, spreading and/or proliferation of cells. These domains and functional analogues of these domains are termed attachment peptides.

Exemplary attachment peptides from fibronectin include, but are not limited to, RGD or Arg Gly Asp (SEQ ID NO:2), REDV or Arg Glu Asp Val (SEQ ID NO:3), and C/H-V (WQPPRARI or Trp Gin Pro Pro Arg Ala Arg Ile) (SEQ ID NO:4).

Exemplary attachment peptides from laminin include, but are not limited to, YIGSR or Tyr Ile Gly Ser Arg (SEQ ID NO:5) and SIKVAV or Ser Ile Lys Val Ala Val (SEQ ID NO:6) and F-9 (RYVVLPRPVCFEKGMNYTVR or Arg Tyr Val Leu Pro Arg Pro Val Cys Phe Glu Lys Gly Met Asn Tyr Thr Val Arg) (SEQ ID NO:7).

Exemplary attachment peptides from collagen include, but are not limited to, HEP-III (GEFYFDLRLKGDK or Gly Glu Phe Tyr Phe Asp Leu Arg Leu Lys Gly Asp Lys) (SEQ ID NO:8) and P15 (GTPGPQGIAGQRGW; SEQ ID NO:1) Desirably, attachment peptides used in this invention have between about 3 and about 30 amino acid residues in their amino acid sequences. Preferably, attachment peptides have not more than about 15 amino acid residues in their amino acid sequences. In one embodiment, attachment peptides have exactly 15 amino acid residues in the amino acid sequences.

An embodiment of the present invention involves synthetic compositions that have a biological activity functionally comparable to that of all or some portion of P15 (SEQ ID NO: 1). By “functionally comparable,” is meant that the shape, size, and flexibility of a compound is such that the biological activity of the compound is similar to the P15 region, or a portion thereof. Biological activities of the peptide may be assessed by different tests including inhibition of collagen synthesis, inhibition of collagen binding, and inhibition of cell migration on a collagen gel in the presence of the peptide in solution. Of particular interest to the present invention is the property of enhanced cell binding. Useful compounds could be selected on the basis of similar spatial and electronic properties as compared to P15 or a portion thereof. These compounds typically will be small molecules of 50 or fewer amino acids or in the molecular weight range of up to about 2,500 daltons, more typically up to about 1000 daltons. Inventive compounds of the invention include synthetic peptides; however, nonpeptides mimicking the necessary conformation for recognition and docking of collagen binding species are also contemplated as within the scope of this invention. For example, cyclic peptides on other compounds in which the necessary conformation is stabilized by nonpeptides (e.g., thioesters) is one means of accomplishing the invention.

The central portion, forming a core sequence, of the P15 region has been identified as having collagen-like activity. Thus, bioactive/biocompatible agents of this Invention may contain the sequence Gly-Ile-Ala-Gly (SEQ ID NO: 9). The two glycine residues flanking the fold, or hinge, formed by -Ile-Ala- are hydrogen bonded at physiologic conditions and thus stabilize the [beta]-fold. Because the stabilizing hydrogen bond between glycines is easily hydrolyzed, two additional residues flanking this sequence can markedly improve the cell binding activity by further stabilizing the bend conformation. An exemplary bioactive/biocompatible agent with advantageous properties contemplated by the present invention, having glutamine at each end (Gln-Gly-Ile-Ala-Gly-Gln; SEQ ID NO: 10) is described in U.S. Pat. No. 6,268,348, issued Jul. 31, 2001, which is incorporated by reference in its entirety herein.

Chemical/biological testing such as MA (amino acid analysis), in vitro cell cultures followed by SEM (scanning electron microscopy), and in vivo testing can be used for evaluating the coatings of the present invention.

A specific example of a coating having biological activity and medical implants having a surface carrying the same and the method incorporating the present invention may now be described as follows.

Let it be assumed that it is desired to coat long porous PTFE tubes, as for example having a length of 11 cm., which are to be utilized as medical implants and to be treated with a coating using the method of the present invention. The tubes can be prepared for treatment by mounting the same on an anodized aluminum wire frame and then inserting them in a vertical position in the upper portion of the plasma chamber being utilized. The tubes are then cleaned in an air plasma by operating the plasma chamber at 0.3 Torr at 50 watts for 3 minutes. After the plasma cleaning operation has been performed, the chamber is flushed with allylamine gas at 0.2 Torr for 10 minutes. Allylamine plasma is then created at 0.2 Torr at 15 watts for 30 minutes. Radiofrequency power is turned off and allylamine is permitted to flow at 0.2 Torr for 2 minutes. The allylamine flow after plasma treatment is provided to react with any free radicals on the PTFE. The allylamine flow is then terminated and a vacuum is maintained in the chamber for 15 minutes. Thereafter, the pressure in the plasma chamber is increased to atmospheric pressure. The tubes being treated are then removed from the chamber and transferred to clean glass rods. The tubes are then submerged and rinsed in an appropriate volume of DMSO. The samples are then removed from the DMSO rinse and washed with deionized (DI) water and optionally ultrasonically at room temperature for 3 minutes.

In the covalent linker attachment step 123, a 1 M (one molar) succinic anhydride solution is prepared using DMSO and placed in a covered glass tray container. The plasma treated and optionally rinsed tubes are then submerged in the succinic anhydride solution in the glass tray container and subjected to an ultrasonic mix at 50° C. in order to bring the succinic anhydride into close proximity to the free amine groups on the PTFE surface. A one molar (1M) Na₂HPO₄ solution in DI water is used to adjust the pH between 6 to 9, preferentially pH 8. A higher pH results in a faster reaction. This reaction between the free amine groups and the succinic anhydride can be carried out between room temperature and 80° C. and preferentially between 20-50° C.

After this has been accomplished, the tubes are removed and rinsed with DI water optionally utilizing ultrasound. The tubes are then dried with nitrogen.

Let it be assumed that a peptide coating is desired to be applied to the surface thus far created. Solubility enhancers such as DMSO and DMF can be added between 0-50 volume/volume v/v %, preferentially 10-30%. A 90 mL. DI water/DMSO solution is prepared by taking 70 mL. of DI water and mixing the same in a glass container with 20 mL. of DMSO. The dried tubes are then placed in the DMSO solution and ultrasonically mixed for a period of 1 minute.

Freshly prepared EDC [N-(3-Dimethylaminopropyl)-N′-ethylcarbodiimide hydrochloride] (Fluka) solution in 5 ml DI water is poured over the tubes submerged in water/DMSO to activate COOH groups on the PTFE surface. After 0.5-3 min., P15 ((H-Gly-Thr-Pro-Gly-Pro-Gln-Gly-Ile-Ala-Gly-Gln-Arg-Gly-Val-Val-OH; SEQ ID NO: 1) acetate salt, GLP grade peptide) solution in 5 ml DI water is added. For hydrophobic peptides, the peptides may be dissolved in an organic solvent miscible with water (DMSO, DMF or dioxane). EDC and P15 amounts are based on the following final concentrations: 0.02 M EDC to be used and 0.0002 M P15 in the final reaction volume, i.e. 100×molar excess of EDC to P15. The reaction at room temperature is carried out between 1-16 hours, preferentially 2-8 hours. The tubes are then rinsed several times with deionized water with an optional one minute ultrasonic treatment. The tubes are then dried with nitrogen gas. The tubes are then inverted to bring the coated side to the inside. Amino acid analysis revealed that up to 1.5 nmol P15/cm² was bound to the PTFE surface.

From the foregoing it can be seen that there has been provided a coating which has biological activities which can be utilized on surfaces of medical implants and devices and a method for accomplishing the same. The coating and method can be utilized on many different types of devices which are intended to be implanted in the human body or in other words to remain in the human body for a period of time. Such devices include stents and grafts placed in various vessels of the human body. Other medical devices such as heart valves, defibrillators and the like have surfaces which are candidates for the coating and method of the present invention. The coating and method is particularly advantageous for use on surfaces which heretofore have been difficult to obtain cell growth on, as for example PTFE and ePTFE. By utilizing the coating and method of the present invention, it has been found that cell growth has been greatly enhanced, making possible long term implantation of said devices in the human body.

Thus the surface of the polymer can be characterized as having applied thereto a bioactive coating which is cell friendly and which enhances growth of cells thereon. As described above, a low temperature plasma-deposited layer is provided on the surface of the polymer to functionalize the surface and provide free amine groups thereon. A spacer/linker molecular layer is covalently bonded to the plasma-deposited layer. A peptide coating such as P15 is deposited on the spacer/linker layer. By way of example, the outer surface of the sleeve 27 can be treated first. Thereafter, the sleeve 27 can be inverted by turning it inside out and treating the inside surface which is now outside. Alternatively, both the outside and inside surfaces can be treated at the same time.

Operation and use of the composite expandable devices 11 and 71 with the delivery apparatus 12 and delivery apparatus 89 may now be briefly described as follows. In this connection let it be assumed that a human heart 101 as shown in FIG. 6 has previously had a coronary artery 102 in which there had been formed therein a substantially total occlusion 103. Also let it be assumed that it was found necessary to perform a bypass operation and to insert a saphenous vein graft utilizing a length of saphenous vein 106 which has one end connected into the aorta 107 of the heart by a proximal anastomosis 108 for a blood supply and bypassing the coronary artery occlusion 103 and making a connection to the coronary artery occlusion 103 and making a connection to the coronary artery 102 at a distal anastomosis 109. Now let it be assumed that after a period of time there has been a build-up of plaque forming a stenosis in the saphenous vein graft 106 in the region near the distal anastomosis 109.

With such a condition, it is desirable to first use a tapered composite expandable device 71, delivering the same by the use of the tapered balloon 86 of the delivery apparatus 89 on a guide wire in a conventional manner through the femoral artery into the aorta, then through the proximal anastomosis 108 and then advanced into a region adjacent the distal anastomosis 109. The distal tapered balloon 86 is then expanded to expand the device 71 into engagement with the wall of the saphenous vein graft and to thereby enlarge the opening through the saphenous vein graft to enhance blood flow therethrough, through the flow passage formed by the device 71. Thereafter, the tapered balloon 86 and the delivery apparatus 89 is removed.

Let it be assumed that the tapered device 71 has an inadequate length to treat the entire stenosis and it is desired to place another composite expandable device as for example the device 11 (FIG. 1) in tandem or in series with the device 71. Assuming that the guide wire is in place that was used for deploying the first device 71, the shaft 14 of the delivery apparatus 12 can be threaded over the guide wire 18 and a balloon with a composite expandable device 11 mounted thereon advanced into the saphenous vein graft 106 until the distal extremity of the device 11 meets within the proximal larger end 77 of the device 71. The distal extremity can be docked into the open proximal end of the device 71. Thereafter, the balloon 13 can be expanded to complete the docking of the distal extremity of the device 11 in the proximal extremity of the device 71 so that they are deployed in the saphenous vein graft 106 in tandem. The balloon 13 then can be deflated and removed with the delivery apparatus 12 along with the guide wire 18. The positioning of the devices 71 and 11 can be observed fluoroscopically by observing the locations of the radiopaque markers 56 provided on the devices 11 and 71. If the occlusion in the saphenous vein graft is sufficiently long, an additional device 11 can be placed in tandem with the device 11 already in place. If this is desired, the guide wire can be left in place and another balloon delivery apparatus 12 with a device 11 mounted thereon can be advanced into the saphenous vein graft 106 and the distal extremity docked into the expanded proximal extremity of the already positioned device 11. The balloon 13 can be deflated and then removed along with the guide wire 18 and the femoral artery closed in an appropriate manner.

From the foregoing it can be seen that the balloon expandable devices 11 and 71 form a vascular prosthesis which has mechanical and biomedical properties which re-establish and mimic the composition of the biological function and environment of a healthy natural vessel as for example a recently transplanted saphenous vein graft. The support frame for the polymer sleeve is designed to provide adequate support for the polymer sleeve while still providing appropriate compliance corresponding to that of the vessel in which it is disposed. The device with its free outer ends is capable of firmly engaging the wall of the vessel in which it is disposed to ensure that the device remains in place in the desired position within the vessel after deployment. By the use of the cylindrical and tapered devices, it is possible to construct a vascular prosthesis which corresponds to the natural geometry of the vessel. The delivery apparatus has a low profile which by utilizing a balloon having an intermediate working portion of a lesser diameter retains this low profile even when the composite expandable device is mounted thereon to facilitate positioning and deployment of the device to the site. Use of the polymer sleeve in the device prevents plaque or deposits within the blood vessel as for example a saphenous vein graft from oozing through the interstices of the frame so that there is unimpeded blood flow through the expanded frame. By covering the polymer sleeve with a peptide such as P15, endothelial cell growth is stimulated. In this way, it is possible to repave the vessel with endothelial cells, nature's most blood compatible surface, and help prevent further spread or degradation of the lumen in the vessel at that site. The construction of the device permitting axial bending makes it possible for the expanded device to readily flex with the vessel.

TABLE 1
Sequence Provided In Support Of The Invention.

| Description | SEQ. ID NO. |
|---|---|
| P15 | 1 |
| GTPGPQGIAGQRGVV | |
| RGD | 2 |
| REDV | 3 |
| C/H-V | 4 |
| WQPPRARI | |
| YIGSR | 5 |
| SIKVAV | 6 |
| F-9 | 7 |
| RYVVLPRPVCFEKGMNYTVR | |
| HEP-III | 8 |
| GEFYFDLRLKGDK | |
| Gly-Ile-Ala-Gly | 9 |
| Gln-Gly-Ile-Ala-Gly-Gln | 10 |

What is claimed:

1. A composite expandable device for delivery into a vessel carrying a blood comprising an expandable support frame having first and second end portions, a polymer sleeve extending over the support frame and having inner and outer surfaces, and a coating disposed on and attached to at least one of the inner and outer surfaces of the polymer sleeve for enhancing endothelial cell growth on the polymer sleeve.

2. A device as in claim 1 wherein said polymer sleeve is porous.

3. A device as in claim 1 wherein said polymer sleeve is impervious.

4. A device as in claim 1 wherein both the inner and outer surfaces are coated with the coating.

5. A device as in claim 1 wherein the first and second end portions are exposed and free of the sleeve.

6. A device as in claim 1 wherein said expandable support frame and polymer sleeve are cylindrical.

7. A device as in claim 1 wherein said expandable support frame and polymer sleeve are tapered.

8. A device as in claim 1 wherein said expandable support frame is constructed to maintain its length during expansion of the frame.

9. A device as in claim 1, wherein said coating is prepared by treating said inner or outer surface with a gaseous plasma cleaning process utilizing radiofrequency energy to ablate said inner or outer surface and to functionalize said inner or outer surface and to produce a plasma-deposited layer having functional groups, and subjecting said plasma-deposited layer to multifunctional linkers/spacers in a wet chemical tratement to form covalent bonds between the linkers/spacers adn the functional groups of the plasma-deptosited layer to covalently bind the cell-adhesion peptides to said inner or outer surface of the substrate.

10. A device as in claim 1 wherein said coating is a cell adhesion peptide.

11. A device as in claim 10 wherein said cell-adhesion peptide has the amino acid sequence presented as SEQ ID NO: 1.

12. A device as in claim 1 wherein said expandable support frame includes a plurality of axially aligned belts and first and second end portions, each of said belts comprising a plurality of circumferentially spaced struts having first and second ends and foldable links secured to the first and second ends of the struts and interconnecting means serially interconnecting the belts and the first and second end portions to extend along an axis and permitting axial bending between the belts and the end portions while maintaining the length of the device.

13. A device as in claim 12 wherein said interconnecting means includes at least one strut and a plurality of S-shaped links.

14. A device as in claim 12 wherein said interconnecting means includes a single strut and first and second S-shaped links, all spaced 120° apart.

15. A device as in claim 12 wherein said interconnecting means between adjacent belts are offset angularly with respect to each other.

16. A device as in claim 15 wherein said end portions are sinusoidal.

17. A device as in claim 12 further including radiopaque markers carried by the end portions.

18. A device as in claim 1 wherein said sleeve is provided with a fold and further including means for securing said label to said sleeve to inhibit dislodging of the sleeve from the frame during deployment of the device.

19. A delivery apparatus for an expandable device having a length and an inner diameter comprising a shaft, a balloon mounted on the shaft, said shaft having a lumen therein for inflating and deflating the balloon, said balloon being formed with proximal, distal and intermediate portions, said intermediate portion having a length to receive the expandable device, and radiopaque markers carried within the proximal and distal portions of the balloon and sized so that they have a diameter greater than the inner diameter of the expandable device when it is mounted on the intermediate portion of the balloon for securing the expandable device to the intermediate portion to prevent the expandable device from being dislodged during deployment by the delivery apparatus, the proximal and distal portions of the balloon being sized so that they have a size which is greater than the size of the expandable device when placed on the intermediate portion to inhibit inadvertent dislodgment of the expandable device during deployment of the expandable device with the apparatus.

20. A method for deploying a plurality of composite expandable devices comprising an expandable frame having opposite ends at proximal and distal extremities, a polymeric sleeve extending over the frame, with the use of a balloon delivery catheter having an inflatable balloon on the distal extremity thereof comprising the steps of mounting a first composite expandable device on the balloon, utilizing the balloon delivery catheter to deliver the device to the desired site in the vessel, inflating the balloon to expand the device in the vessel, deflating the balloon and removing the balloon from the vessel, utilizing a balloon delivery catheter to deliver a second composite expandable device to the site and docking the distal extremity of the additional composite expandable device in the proximal extremity of the first composite device already in place by causing the extremities to intermesh with each other, expanding the balloon to expand the second composite expandable device to expand the distal extremity within the proximal extremity of the composite expandable device already in place to complete the docking and deflating the balloon and removing the balloon delivery catheter from the vessel.

21. A method as in claim 20 wherein the first device is a tapered device.

22. A device for delivery into a vessel carrying a blood comprising a polymer sleeve having inner and outer surfaces, and a coating disposed on and attached to at least one of the inner and outer surfaces of the polymer sleeve for enhancing endothelial cell growth on the polymer sleeve.


SEQUENCE LISTING (10 sequences)

SEQ ID NO: 1 - protein, 15 aa, Artificial Sequence
  attachment peptide from collagen
GTPGPQGIAGQRGVV

SEQ ID NO: 2 - protein, 3 aa, Artificial Sequence
  attachment peptide from fibronectin
RGD

SEQ ID NO: 3 - protein, 4 aa, Artificial Sequence
  attachment peptide from fibronectin
REDV

SEQ ID NO: 4 - protein, 8 aa, Artificial Sequence
  attachment peptide from fibronectin
WQPPRARI

SEQ ID NO: 5 - protein, 5 aa, Artificial Sequence
  attachment peptide from laminin
YIGSR

SEQ ID NO: 6 - protein, 6 aa, Artificial Sequence
  attachment peptide from laminin
SIKVAV

SEQ ID NO: 7 - protein, 20 aa, Artificial Sequence
  attachment peptide from laminin
RYVVLPRPVCFEKGMNYTVR

SEQ ID NO: 8 - protein, 13 aa, Artificial Sequence
  attachment peptide from collagen
GEFYFDLRLKGDK

SEQ ID NO: 9 - protein, 4 aa, Artificial Sequence
  bioactive/biocompatible agent
GIAG

SEQ ID NO: 10 - protein, 6 aa, Artificial Sequence
  bioactive/biocompatible agent
QGIAGQ